(19) United States
(12) Patent Application Publication
(10) Pub. No.: US 2026/0086631 A1
(43) Pub. Date: Mar. 26, 2026

(54) SYSTEMS AND METHODS FOR SETTING BOOKMARK MOMENTS AND CONTROLLING MEDIA

(71) Applicant: GM GLOBAL TECHNOLOGY OPERATIONS LLC, Detroit, MI (US)

(72) Inventors: Eric T. Hosey, Rochester Hills, MI (US); Matthew Edward Gilbert-Eyres, Rochester Hills, MI (US); Russell A. Patenaude, Macomb Township, MI (US); Benjamin Tran, Royal Oak, MI (US)

(21) Appl. No.: 18/894,963

(22) Filed: Sep. 24, 2024

(51) Int. Cl. G06F 3/01; A61B 5/16; G06F 3/0482; G06F 3/0484; A61B 5/0205

(52) U.S. Cl. CPC G06F 3/013; A61B 5/163; A61B 5/165; G06F 3/0482; G06F 3/0484; A61B 5/0205

(57) ABSTRACT

A system for controlling media includes one or more sensors configured to detect one or more characteristics and a control module. The control module is configured to detect whether a user is observing media output by a media output module based on at least one of the detected characteristics, in response to detecting the user is observing the media output by the media output module, determine a focus level of the user based on at least one of the detected characteristics, set at least one bookmark moment with respect to the media based on the focus level of the user, and output one or more resumption options for the user with respect to the media, the one or more resumption options including reverting to the at least one bookmark moment. Other example systems and methods for controlling media are also disclosed.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to systems and methods for setting bookmark moments and controlling media.

Media, such as audio, video, etc. may be presented in various environments. For example, users may observe movies, shows, music, podcasts, etc. while in a stationary environment (e.g., a building, a house, etc.) or in a movable environment (e.g., in a vehicle, etc.). Depending on the type of media and/or the medium providing the media, a user may select an input to rewind or forward the media in play time. For instance, the input may rewind or forward the media in a defined chunk of time (e.g., 15 seconds per input selection). In other examples, the input may rewind or forward the media continuously while the input is selected.

### SUMMARY

A system for controlling media includes one or more sensors configured to detect one or more characteristics, and a control module in communication with the one or more sensors. The control module is configured to detect whether a user is observing media output by a media output module based on at least one of the detected characteristics, in response to detecting the user is observing the media output by the media output module, determine a focus level of the user based on at least one of the detected characteristics, set at least one bookmark moment with respect to the media based on the focus level of the user, and output one or more resumption options for the user with respect to the media, the one or more resumption options including reverting to the at least one bookmark moment.

In other features, the control module is configured to pause the media output by the media output module in response to the focus level of the user indicating the user is inattentive or absent.

In other features, the control module is configured to receive a user request to resume the media and in response to receiving the user request, output the one or more resumption options for the user with respect to the media.

In other features, the user is a first user and the bookmark moment is a first bookmark moment specific to the first user, and the control module is configured to determine a focus level of a second user observing the media based on at least one of the detected characteristics.

In other features, the control module is configured to set a second bookmark moment specific to the second user with respect to the media based on the focus level of the second user and output the one or more resumption options with respect to the media, the one or more resumption options including reverting to the first bookmark moment and the second bookmark moment.

In other features, the control module is configured to prompt the second user to determine if the second user would like to continue observing the media in response to the focus level of the second user indicating the second user is attentive or present.

In other features, the control module is configured to receive feedback from the second user and continue playing or pause the media output by the media output module in response to the feedback from the second user.

In other features, the control module is configured to determine whether the media is pauseable prior to detecting whether the user is observing the media.

In other features, the one or more sensors include at least one of an occupancy sensor, a motion sensor, a camera, a radar sensor, a biometric sensor, and a microphone.

In other features, the one or more detected characteristics include one or more of a location of the user, an eye focus of the user, a paired output device, and speaker tuning, and the control module is configured to detect whether the user is observing media output by the media output module based on one or more of the location of the user, the eye focus of the user, the paired output device, and the speaker tuning.

In other features, the one or more detected characteristics include one or more of an eye focus of the user, movement of the user, biometric data specific to the user, and environment data, and the control module is configured to determine the focus level of the user based on one or more of the eye focus of the user, the movement of the user, the biometric data specific to the user, and the environment data.

In other features, the focus level of the user is one of a plurality of focus states, and the control module is configured to determine a focus state from the plurality of focus states for the user based on the at least one of the detected characteristics and set the at least one bookmark moment in response to the determined focus state.

In other features, the plurality of focus states include a focused and observable state, an inattentive and observable state, and a non-observable state.

In other features, the set bookmark moment is one of a plurality of set bookmark moments for the media, and the one or more resumption options include reverting to the set bookmark moment, implementing a contextual search of the plurality of set bookmark moments or the media, and displaying a menu of the plurality of bookmark moments.

In other features, the one or more sensors and the control module are positioned in a vehicle.

A method for controlling media includes receiving, from at least one sensor, one or more characteristics, detecting whether a user is observing media output by a media output module based on at least one of the characteristics, in response to detecting the user is observing the media output by the media output module, determining a focus level of the user based on at least one of the characteristics, setting at least one bookmark moment with respect to the media based on the focus level of the user, and outputting one or more resumption options for the user with respect to the media, the one or more resumption options including reverting to the at least one bookmark moment.

In other features, the method further includes pausing the media output by the media output module in response to the focus level of the user indicating the user is inattentive or absent.

In other features, outputting the one or more resumption options for the user includes outputting the one or more resumption options in response to receiving a user request to resume the media.

In other features, the user is a first user, and the method further include determining a focus level of a second user observing the media based on at least one of the characteristics, and prompting the second user to determine if the second user would like to continue observing the media in response to the focus level of the second user indicating the second user is attentive or present.

A vehicle system for controlling media includes one or more sensors positioned in a vehicle, the one or more sensors configured to detect one or more characteristics, and a control module in communication with the one or more sensors. The control module configured to detect whether a user is observing media output by a media output module based on at least one of the detected characteristics, in response to detecting the user is observing the media output by the media output module, determine a focus state from a plurality of focus states for the user based on at least one of the detected characteristics, set at least one bookmark moment with respect to the media based on the determined focus state for the user, and output one or more resumption options for the user with respect to the media, the one or more resumption options including reverting to the at least one bookmark moment.

In other features, the plurality of focus states include a focused and observable state, an inattentive and observable state, and a non-observable state.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a block diagram of an example system for controlling media, according to the present disclosure;

FIG. 2 is a vehicle including portions of the system of FIG. 1, according to the present disclosure; and

FIGS. 3-5 are flowcharts of example control processes for controlling media, according to the present disclosure.

In the drawings, reference numbers may be reused to identify similar and/or identical elements.

### DETAILED DESCRIPTION

Media, such as movies, shows, songs, podcasts, etc. may be presented in various environments. A user often becomes frustrated in attempting to find a moment in the media when the user fell asleep, exited a particular environment (e.g., exited a room, exited a vehicle, etc.), started paying attention to something else entirely, etc. Depending on the viewing/listening medium employed, accomplishing this task can also be difficult and even dangerous. For example, in a vehicle while listening to a podcast, a user (e.g., a driver, a passenger, etc.) may be limited to rewinding in 15 second chunks or rewinding continuously to aimlessly find a moment in the media where the user fell asleep, exited the vehicle, became distracted, etc. In such examples, the user is either stuck pressing an input bottom multiple times or continuously for an extended period of time, causing the user to shift focus from the road and its surroundings to the input bottom and the viewing/listening medium. This shifted focus dangers the user (e.g., the driver), passengers in the vehicle, other drivers and/or pedestrians. While the level of endangerment may be less in non-vehicle environments or non-driving vehicle environments, user frustration remains high due to the aimless control of media resumption options.

The systems and methods according to the present disclosure provide solutions for automatically setting bookmark moments with respect to media being played to enable users to conveniently revert to previous moments in the media. For example, the systems and methods provide for the detection of users observing media, the detection of when focus levels or states of users indicate inattentiveness (e.g., distraction, not paying attention, asleep, etc.) and/or absence, and the setting of bookmark moments with respect to the media based on the focus levels or states. In various embodiments, the systems and methods may automatically pause the media at that the bookmark moment and/or provide one or more resumption options, thereby ensuring the users do not lose their place and can easily resume. As a result, the level of endangerment due to the lack of focus and the level of frustration of users trying to revert to particular moments in consumed media may be reduced.

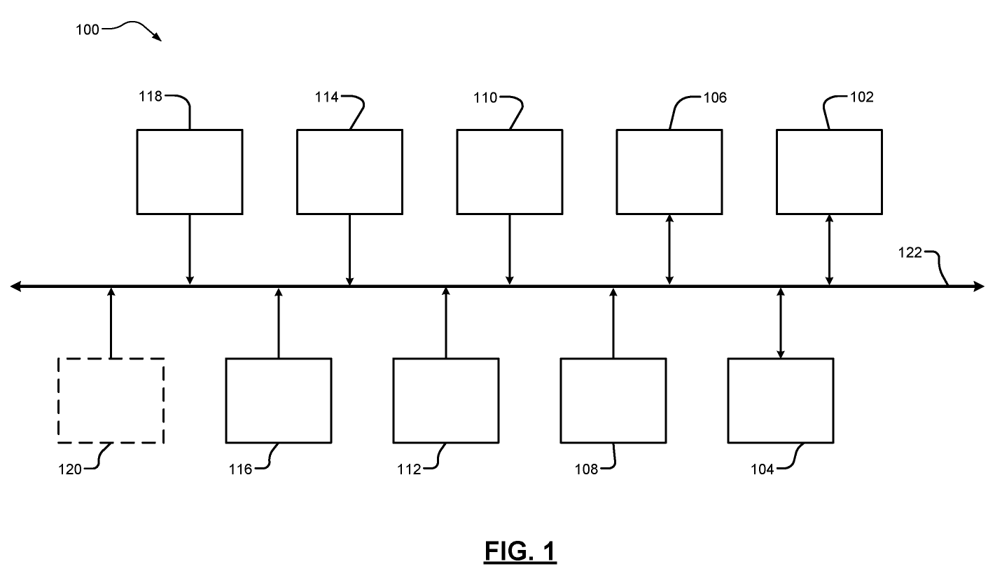

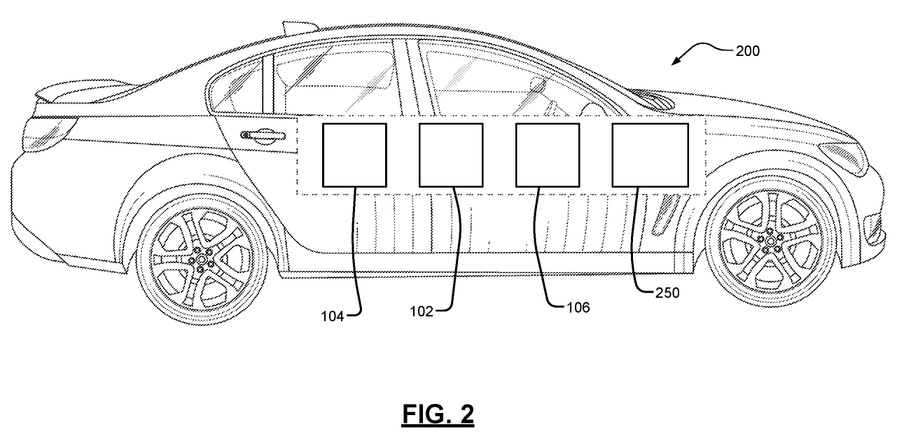

Referring now to FIG. 1, a block diagram of an example system 100 is presented for controlling media. As shown in FIG. 1, the system 100 generally includes a control module 102, a media output module 104, a notification module 106, and various sensors. In the example of FIG. 1, the sensors may include one or more occupancy detection sensors, one or more user tracking sensors, one or more microphones 118, and/or one or more vehicle sensors 120. The occupancy detection sensors include, for example, one or more occupancy sensors 108, biometric sensors 112, motion sensors 116, and the tracking sensors include, for example, one or more eye tracker sensors 110 and face tracker sensors 114.

Although FIG. 1 illustrates the system 100 as including specific modules, it should be appreciated that one or more other modules may be employed if desired. Additionally, while the system 100 is shown as including multiple separate modules, any combination of the modules and/or the functionality thereof may be integrated into one or more modules. Further, although the system 100 of FIG. 1 is shown as including particular sensors, it should be appreciated that the system 100 and/or other systems may include more or less sensors, sensors having different functionalities, etc.

In various embodiments, the modules and sensors of the system 100 may be in communication with each other and may share parameters via a network 122, such as a wired network (e.g., a controller area network (CAN), etc.), a wireless network, etc. In such examples, the parameters may be shared via one or more data buses of the network 122. As such, various parameters may be made available by a given module and/or sensor to other modules and/or sensors via the network 122.

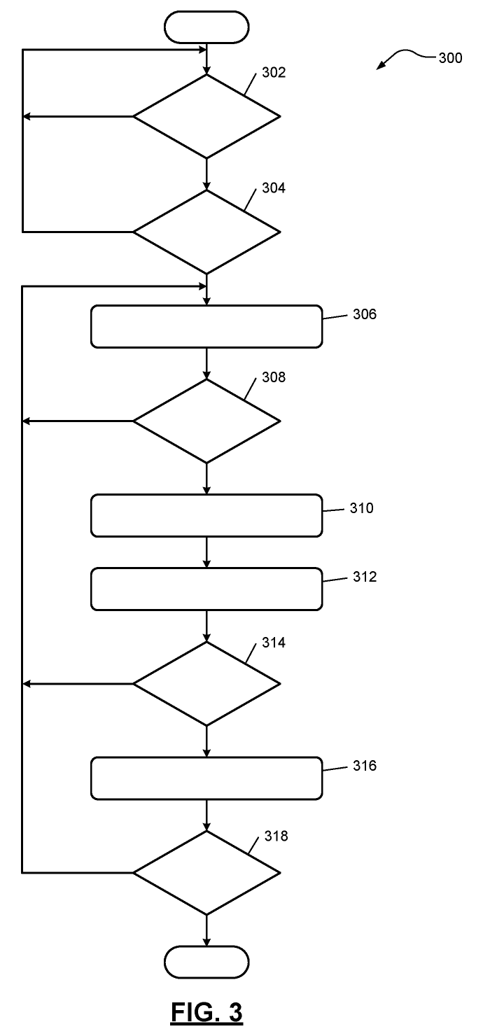

The system 100 of FIG. 1 may be employable in any suitable environment. For example, the system 100 may be employed in a vehicle, such as an electric vehicle (e.g., a pure electric vehicle, a plug-in hybrid electric vehicle, etc.), an internal combustion engine vehicle, etc. Additionally, the system 100 may be applicable to an autonomous vehicle, a semi-autonomous vehicle, etc. For example, FIG. 2 depicts a vehicle 200 including the control module 102, the media output module 104, and the notification module 106 of FIG. 1, and one or more sensors 250 (e.g., any one or more of the sensors of FIG. 1, etc.) in communication with the control module 102. While the system 100 is generally described herein relative to a vehicle (e.g., the vehicle 200), it should be appreciated that the system 100 may be implemented with other suitable environments, such as other modes of transportation, in rooms (e.g., living rooms, bedrooms, waiting rooms, etc.), etc.

In various embodiments, the media output module 104 may be any suitable device for outputting media, such movies, shows, songs, podcasts, etc. For example, the media output module 104 may include one or more speakers for providing audio for a podcast played on a podcast application and/or one or more displays for providing video for a show, movie, etc.

The notification module 106 of FIG. 1 generally outputs one or more notifications for users. For example, and as further explained below, the notification module 106 may display visual notifications, provide audible notifications, etc. of one or more requests for user input, resumption options for selection, etc. As such, in various embodiments, the notification module 106 may include one or more displays, protection devices, speakers, etc.

In various embodiments, the occupancy detection sensors of FIG. 1 generally detect or sense a parameter associated with locations of users. For example, in a vehicle, the occupancy detection sensors may detect or sense whether a seat is occupied. As example only, the occupancy sensors 108 may detect a weight placed on a seat, such as the weight of a user, etc. As such, the occupancy sensors 108 may be weight sensors positioned in seats for sensing mass. In other examples, the occupancy sensors 108 may be cameras, radar sensors, etc. for sensing sizes and/or shapes of objects (e.g. users). Additionally, the biometric sensors 112 may include heart rate sensors, respiration sensors, and/or another suitable sensor positioned in a seat for detecting a biometric (e.g., a heart rate, breathing, etc.) of an occupant. Further, the motion sensors 116 may include, for example, radar sensors, cameras, etc. for detecting motion in the environment (e.g., a user exiting a vehicle, a user exiting a room, etc.).

The user tracking sensors of FIG. 1 generally detect or sense whether an occupant is observing media or looking in the direction of the media output module 104. For example, the eye tracker sensors 110 may include cameras and/or radar sensors for tracking the occupant’s eye, and the face tracker sensor 114 may include and/or radar sensors for tracking movements of the occupant’s face.

The vehicle sensors 120 of FIG. 1 may be employed in vehicle applications. For example, the vehicle sensors 120 may include any suitable sensors for collecting vehicle data that may be indicative of user distractions, such as near collisions moments, heavy traffic detections, navigation route events, etc. In examples in which the system 100 is employed in non-vehicle environments, the vehicle sensors 120 may be replaced with sensors to generally collect data related to events that cause user distractions. In other examples, the vehicle sensors 120 may be omitted.

With continued reference to FIG. 1, the control module 102 may receive data from one or more of the sensors. In such examples, the received data may be or represent various characteristics associated with one or more users in the environment (e.g., the vehicle 200, a room, etc.), the media playing, the environment, etc. For example, user characteristics may include eye features, facial features, movement, presence, etc. Media characteristics may include, for example, characteristics indicating whether the media is cable of being paused, whether the media is playing, etc. Environmental characteristics may include, for example, vehicle events (e.g., abrupt deacceleration, abrupt lateral movement, vehicle impact, other media playing, connected devices, traffic congestion, upcoming vehicle maneuvers, etc.), room events (e.g., darkness, other media playing, connected devices, etc.), ambient noises detected by the microphone(s) 118, etc.

In various embodiments, the control module 102 may optionally detect all currently playing media. In such examples, the control module 102 may identify a location of the media output module 104 playing the media, such as in a rear portion of the vehicle 200, a front portion of the vehicle 200, a driver side rear or front portion of the vehicle 200, a passenger side rear or front portion of the vehicle 200, etc. Additionally, the control module 102 may optionally determine whether the media is pauseable. For instance, some types of media such as FM broadcasting, AM broadcasting, satellite broadcasting, non-streaming media, etc. are not capable of being paused, while other types of media such as podcasts, movies, streaming media, etc. are capable of being paused. This determination may be made prior to detecting whether the user or users are observing the same media or different media. In such examples, the control module 102 may receive a signal from the media output module 104 and/or other media output modules indicating the playing of media and control options for that media, such as the possibility of being paused (e.g., includes a pause input, etc.).

In various embodiments, the control module 102 detects a user observing the media output by the media output module 104. In some examples, the control module 102 may detect one user listening, watching, or otherwise observing one type of media. In other examples, the control module 102 may detect multiple users listening observing the same media or different media. For example, if multiple users are present and/or multiple media options are provided, the control module 102 may determine which specific users are observing which specific media output by the media output module 104 and/or other media output modules.

The control module 102 may make this determination based on at least one of the detected characteristics. For instance, the detected characteristics may include the location of a user, an eye focus of a user, a paired output device, speaker tuning, etc. As examples only, the control module 102 may rely on data provided by the occupancy sensor(s) 108 and the motion sensor(s) 116 to detect seat locations in relation to the media output module 104 and/or other media output modules. Additionally, in some examples, the control module 102 may rely on data from cameras (e.g., in the eye tracker sensor(s) 110, the face tracker sensor(s) 114, etc.) to detect user focus on specific a location, such as the media output module 104 and/or other media output modules. Further, in other examples, the control module 102 may rely on data indicating the usage of paired output devices (e.g., wirelessly paired headphones, auxiliary devices, phones, etc.), the specific tuning of speakers (e.g., in the vehicles 200, etc.) in relation to the users.

Additionally, the control module 102 may rely on different characteristics for different types of media. For example, for visual media, the control module 102 may consider the location of observers in relation to a screen and use cameras to detect eye focusing. However, for audio media, the control module 102 may consider the location or tuning of speakers in relation to the observers, as well as the use of paired output devices. In other examples where the media is a combined audio and video presentation, the control module 102 may employ a combination of characteristics to detect specific users.

In response to detecting one or more users observing the same or different media, the control module 102 determines a focus level of each of the detected users. For example, the control module 102 may rely on at least one of the detected characteristics to make this determination. For instance, the control module may determine the focus level of a user based on the eye focus of that user, the movement of that user, the biometric data specific to the user, environment data (e.g., vehicle data, etc.) etc. As examples only, the control module 102 may rely on data provided by cameras, radar sensors, etc. to detect a level of eye focus, distractions that have entered an observable area for the user, a sleep state, etc. Additionally, the control module 102 may rely on data provided by the biometrics sensor(s) 112 to detect focus and sleep state, the motion sensor(s) 116 to detect if observers have left an observable area, the motion sensor(s) 116 to monitor entry and exit points of the observable area, etc. Further, the control module 102 may rely on data provided by the microphone(s) 118 to detect distractions or interruptive ambient noise and/or the vehicle sensor(s) 120 to determine distractions (e.g., near collisions, traffic detection, navigation route event, etc.).

In various embodiments, the focus level may be associated or otherwise indicate a status specific to a user. For instance, a focus level for one user or observer may be the same or different for another user or observer. In such examples, the focus level may indicate inattentiveness and presence, or absence (or not present). For example, inattentiveness of a user may be that the user is distracted, not paying attention, asleep, etc., presence of a user may be that the user is in an observable range/area of the media output module 104 and/or another media output module, and absence of a user may be that the user is not in an observable range/area of the media output module 104 and/or another media output module.

In some examples, the focus level for a user may include a score, such as a weighted score. In such examples, the control module 102 may determine a score specific to a user based on the detected characteristic(s). This score may be weighted as desired based on the importance of the characteristic(s). Then, the control module 102 may compare the score or a representative thereof to one or more thresholds to determine a focus level of the user.

In other examples, the focus level for a user may be one of multiple different focus states. In such examples, the control module 102 may determine a focus state (or the multiple different focus states) for a specific user based on the detected characteristic(s), as explained above. In various embodiments, the focus states may include a focused and observable state, an inattentive and observable state, and a non-observable state. In such examples, the focused and observable state includes instances where the user is both focused on the playing media and present (e.g., in an observable range/area of the media output module 104 and/or another media output module). The inattentive and observable state includes instances where the user is present but is distracted, not paying attention, asleep, etc. The non-observable state includes instances where the user is not in an observable range/area of the media output module 104 and/or another media output module.

Then, the control module 102 sets one or more bookmark moments with respect to the media based on the focus level of at least one user. For example, if a focus level for one user indicates inattentiveness or absence (e.g., in an inattentive and observable state, a non-observable state, etc.), the control module 102 may automatically set a bookmark moment with respect to the media for that observing user. Additionally, if a focus level for a second user indicates inattentiveness or absence (e.g., in an inattentive and observable state, a non-observable state, etc.), the control module 102 may automatically set a bookmark moment with respect to the same or different media the second user is observing. As such, the control module 102 may set different bookmarks moments specific to different users for the same or different media being played.

In such examples, each bookmark moment may be tied to a point in time of the media being played. For example, the control module 102 may set each bookmark moment by setting a flag at a particular timestamp of the media being played. The set flags may be stored in memory for any suitable period of time, including after the media being played is manually paused via user input, stopped, etc.

In some examples, one or more users may set up user profiles specific to the users. In such examples, each user may adjust, define, etc. weighting factors for determining a focus level specific to the user, sensor sensitivity for the user, etc. Further, when bookmark moments are set, the control module 102 may tie particular ones to a user profile. Then, if desired, the control module 102 can recall specific bookmark moments tied to a user profile when resumption options are provided.

In various embodiments, the control module 102 may pause the media output by the media output module 104 and/or another media output module in response to the focus level of at least one user indicating the user is inattentive or absent. For example, if the control module 102 determines a first user is inattentive and/or absent (e.g., in an inattentive and observable state, a non-observable state, etc.), the control module 102 may automatically pause the media observed by that user, thereby ensuring the user (or observer) does not lose his/her place and can easily resume. In such examples, the control module 102 may transmit a control signal to the media output module instructing it to pause the media.

In other examples, the control module 102 may pause the media only if no other user observing the media has a focus level indicating attentiveness and presence, or if another user approves of the pausing. For example, in some embodiments where the focus level of a first user or observer indicates that user is inattentive or absent, the control module 102 may prompt a second user or observer to determine if the second user would like to continue observing the media. This prompt may be provided if the control module 102 determines that a focus level for the second user indicates attentiveness and/or presence. In such examples, the prompt may be a visual notification, an audible notification. etc. provided by the notification module 106.

Then, based on feedback received from the second user, the control module 102 may proceed to pause or not pause the media. For instance, if the second user wishes to continue playing the media as indicated by the received feedback, the control module 102 may store the set bookmark moments for the first user and continue playing the media. If, however, the second user wishes to pause the media, the control module 102 may store the set bookmark moments for the first user and pause the media. Such user feedback may be provided via selection on a user interface, vocal commands, etc.

In various embodiments, the control module 102 may also output one or more resumption options for any one of the users with respect to the media currently being or previously observed. The resumption option(s) may be provided regardless of whether the media is paused, stopped, playing, concluded, etc. In such examples, the resumption option(s) may include an option of reverting to any one of the set bookmark moments for one particular user or the collection of users. For instance, the resumption option(s) may include options of reverting to one or more set bookmark moments for a first user, options of reverting to one or more set bookmark moments for a second user, etc. In such examples, a user may select one of the resumption option(s) for the media, which is provided to the control module 102 for execution.

In some examples, the resumption option(s) may include other suitable options for the media. For instance, in some examples, the resumption option(s) may include looping back to any previous set bookmark moment regardless of being tied to a specific user/observer and/or displaying a menu (e.g., on the notification module 106) of different set bookmark moments. Additionally, in some examples, the resumption option(s) may include options for contextual searches. For instance, the resumption option(s) may provide selectable options for contextual searches of the set bookmark moments and/or contextual searches of media where no bookmark moments are found.

The control module 102 may output the resumption option(s) in response to any suitable triggering event. For example, the control module 102 may automatically output the resumption option(s) in response to a defined number of set bookmark moments. In other examples, the control module 102 may output the resumption option(s) in response to a user request. In such examples, the control module 102 may receive a user request to resume the media (e.g., via selection on a user interface, vocal commands, etc.), and then in response to receiving the user request, output the resumption option(s).

In other examples, the control module 102 may automatically restart the media if one or more conditions apply. For example, if the media is paused, the control module 102 may automatically restart the media if the focus level of the user returns to attentiveness and presence, regardless of whether a resumption option has been provided and/or user feedback has been received. In such examples, the control module 102 may restart the media at the latest set bookmark moment or another suitable timestamp.

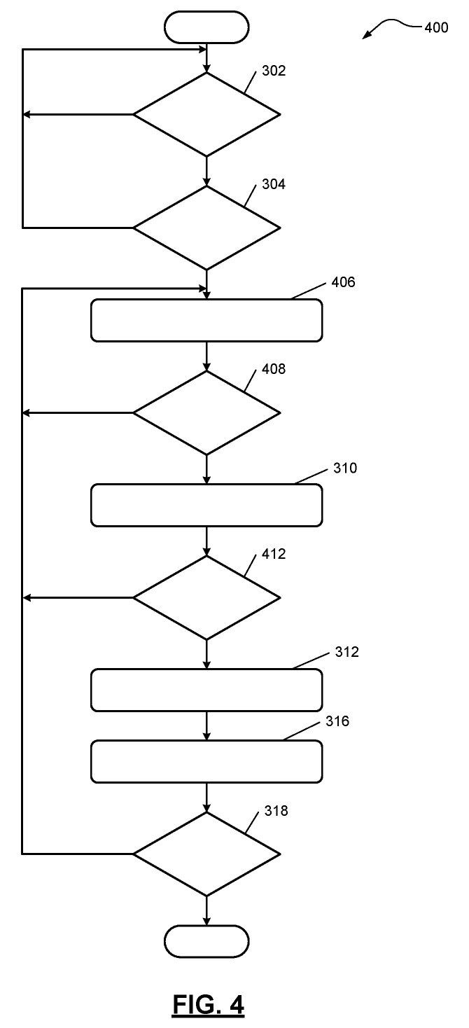

As shown in FIG. 3, the control process 300 begins at 302 by determining whether any media is playing. In such examples, the control module 102 may receive a signal from the media output module 104, receive data from one or sensors (e.g., the microphone(s) 118), etc. indicating that media is playing. If no, the control process 300 returns to 302. If yes, the control process 300 proceeds to 304 where the control module 102 determines if any users are observing the media. For example, the control module 102 may make this determination based on detected characteristics, such as the location of a user, an eye focus of a user, a paired output device, speaker tuning, etc. as explained above. If no at 304, the control process 300 returns to 302. If yes, the control process 300 proceeds to 306.

At 306, the control module 102 determines a focus level of each user observing the media. For example, and as explained above, the control module 102 may make this determination based on detected characteristics, such as the eye focus of that user, the movement of that user, the biometric data specific to the user, environment data (e.g., vehicle data, etc.) etc. In such examples, the focus level for a user may indicate the inattentiveness and presence of the user or an absence of the user. The control process 300 then proceeds to 308.

At 308, the control module 102 determines whether the focus level for a particular user is below a threshold. For example, the control module 102 may calculate or assign a score indicative of the focus level for each user observing the media, and then compare the score(s) score to the threshold. If the score (and therefore the focus level) is greater than or equal to the threshold (e.g., indicating the user is attentive), the control process 300 returns to 306. Otherwise, if the score (and therefore the focus level) is less than the threshold (e.g., indicating the user is inattentive and/or absent), the control process 300 proceeds to 310.

At 310, the control module 102 sets a bookmark moment with respect to the media for the user. For example, and as explained above, the bookmark moment may be a flag or the like set at a particular timestamp of the media being played. In such examples, the bookmark moment, the flag and/or the timestamp may be stored in memory for future use. The control process 300 then proceeds to 312, where the control module 102 outputs one or more resumption options for the user. For example, the resumption option(s) may include selectable options of reverting to a set bookmark moment (e.g., in a displayed menu, etc.), contextual searching of multiple set bookmark moments, etc. The control process 300 then proceeds to 314.

At 314, the control module 102 determines whether any of the provided resumption options is selected for execution. For example, the user may select one of the provided resumption option(s) via a user interface, a vocal command, etc. if no resumption option is selected, the control process 300 returns to 306. If, however, the control module 102 determines that a resumption option is selected, the control process 300 proceeds to 316 where the control module 102 implements the selected option. In such examples, the control module 102 may transmit a control signal to the media output module 104 based on the selected option, thereby instructing the media output module 104 to revert back to a particular bookmark moment.

The control process 300 then proceeds to 318. At 318, the control module 102 determines whether the media output module 104 is still activated (e.g., powered on, etc.) and playing the media. For example, if implemented in the vehicle 200, the media output module 104 may be turned off, shut down, powered off, etc. manually and/or when the vehicle is powered off. If yes at 318, the control process 300 returns to 306. Otherwise, if no at 318, the control process 300 may end as shown in FIG. 3.

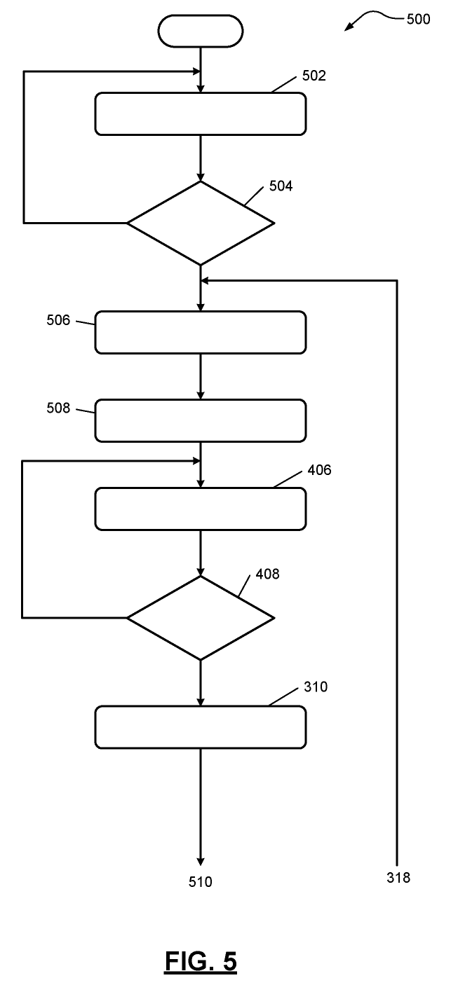

In FIG.4, the control process 400 is similar to the control process 300 of FIG. 3 but includes alternative and/or additional steps. For example, and as shown in FIG. 4, the control process 400 begins at 302 of FIG. 3 as explained above, and then proceeds to 304 of FIG. 3 and 406. At 406, the control module 102 determines a focus state from a set of defined focus states for each user observing the media. For example, and as explained above, the control module 102 may make this determination based on detected characteristics, such as the eye focus of that user, the movement of that user, the biometric data specific to the user, environment data (e.g., vehicle data, etc.) etc. In such examples, the focus states may include a focused and observable state, an inattentive and observable state, and a non-observable state. The control process 400 then proceeds to 408.

At 408, the control module 102 determines whether the determined focus state for the user is indicative of inattentiveness or absence. As one example, if the determined focus state is the focused and observable state (e.g., the determined focus state is not indicative of inattentiveness or absence), the control process 400 returns to 406. If, however, the determined focus state is one of the inattentive and observable state or the non-observable state (e.g., the determined focus state is indicative of inattentiveness or absence), the control process 400 proceeds to 310 of FIG. 3 where a bookmark moment is set as explained above. The control process 400 then proceeds to 412.

At 412, the control module 102 determines whether a request to alter the media has been received. For example, and as explained above, the control module 102 may receive a request from a user to resume the media (if previously paused, stopped, etc.) via selection on a user interface, vocal commands, etc. If no at 412, the control process 400 returns to 406. If yes at 412, the control process 400 proceeds to 312, 316, 318 of FIG. 3 explained above.

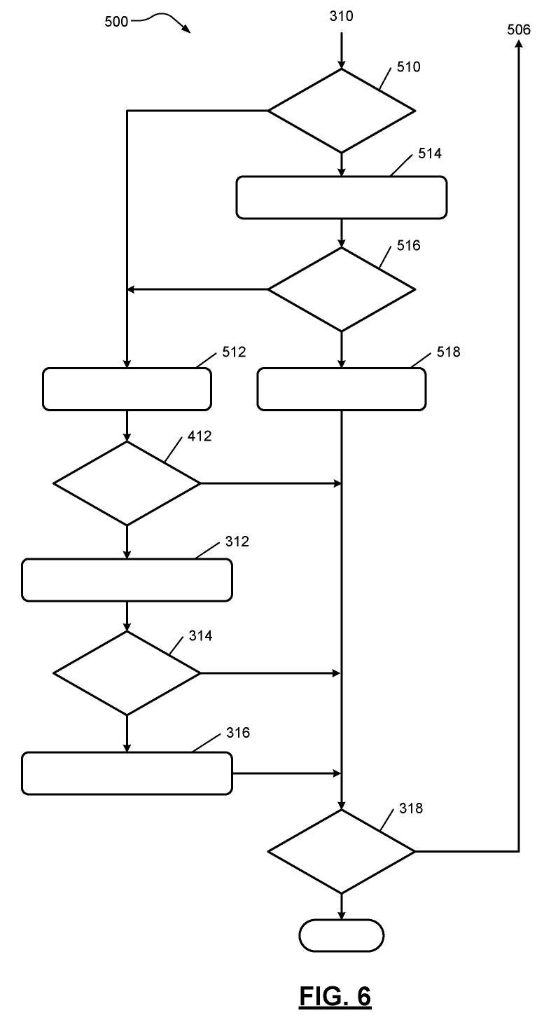

In FIGS. 5-6, the control process 500 is similar to the control processes 300, 400 of FIGS. 3-4 but includes alternative and/or additional steps. For example, in FIG. 5, the control process 500 begins at 502 where the control module 102 detects all currently playing media and locations thereof. In such examples, the control module 102 may receive a signal from the media output module 104 and/or other media output modules playing media, receive data from one or sensors (e.g., the microphone(s) 118), etc. indicating that media is playing. The control process 500 then proceeds to 504.

At 504, the control module 102 determines whether the detected media can be paused. For example, and as explained above, the control module 102 may receive a signal from the media output module 104 and/or other media output modules indicating control options for that media, such as the possibility of being paused. If no at 504, the control process 500 returns to 502. If yes at 504, the control process 500 proceeds to 506 where the control module 102 receives or otherwise access detected characteristics associated with the users, the environment, etc., such as locations of users, eye focus of users, movement of users, paired output devices, speaker tuning, etc. The control process 500 then proceeds to 508.

At 508, the control module 102 determines which specific users are observing which specific media output by the media output module 104 and/or other media output modules. As explained above, the control module 102 may make this determination based on at least one of the detected characteristics. The control process 500 then proceeds to 406 of FIG. 4 where the control module 102 determines a focus state from a set of defined focus states for each user observing the media and then to 408 of FIG. 4 where the control module 102 determines whether the determined focus state for the user is indicative of inattentiveness or absence. If no at 408, the control process 500 returns to 406. If yes at 408, the control process 500 proceeds to 310 of FIG. 3 explained above. The control process 500 then proceeds to 510, as shown in FIG. 6.

At 510, the control module 102 determines whether any other focused (and present) users are observing the media. This determination may be made based on at least one of the detected characteristics, as explained above. If no at 510, the control process 500 proceeds to 512 where the control module 102 pauses the media as explained herein. Otherwise, if other focused users are observing the media (as determined at 510), the control process 500 proceeds to 514.

At 514, the control module 102 prompts the other focused users to determine if the users would like to continue observing the media. This prompt may be a visual notification, an audible notification. etc. provided by the notification module 106, as explained above. The control process 500 then proceeds to 516, where the control module 102 determines whether to continue playing the media. For example, and as explained above, any one of the users may respond to the prompt with an input to continue or pause the media.

If yes at 516, the control process 500 proceeds to 518 where the control module 102 continues playing the media (or does not attempt to pause the media). The control process 500 then proceeds to 318 of FIG. 3, as explained above. If yes at 318, the control process 500 returns to 506. If no at 318, the control process 500 may end as shown in FIG. 6.

If no at 516, the control process 500 proceeds to 512 where the control module 102 pauses the media. The control process 500 then proceeds to 412, 312, 314, 316, 318 of FIGS. 3 and 4, as explained above. For example, if no at 412 or 314 in FIG. 6, the control process 500 proceeds to 318. However, if yes at 412, the control process 500 proceeds to 318, and if yes at 314, the control process 500 proceeds to 316.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A system for controlling media, the system comprising:
one or more sensors configured to detect one or more characteristics; and
a control module in communication with the one or more sensors, the control module configured to:
detect whether a user is observing media output by a media output module based on at least one of the detected characteristics;
in response to detecting the user is observing the media output by the media output module, determine a focus level of the user based on at least one of the detected characteristics;
set at least one bookmark moment with respect to the media based on the focus level of the user; and
output one or more resumption options for the user with respect to the media, the one or more resumption options including reverting to the at least one bookmark moment.

2. The system of claim 1, wherein the control module is configured to pause the media output by the media output module in response to the focus level of the user indicating the user is inattentive or absent.

3. The system of claim 2, wherein the control module is configured to:
receive a user request to resume the media; and
in response to receiving the user request, output the one or more resumption options for the user with respect to the media.

4. The system of claim 1, wherein:
the user is a first user and the bookmark moment is a first bookmark moment specific to the first user; and
the control module is configured to determine a focus level of a second user observing the media based on at least one of the detected characteristics.

5. The system of claim 4, wherein the control module is configured to:
set a second bookmark moment specific to the second user with respect to the media based on the focus level of the second user; and
output the one or more resumption options with respect to the media, the one or more resumption options including reverting to the first bookmark moment and the second bookmark moment.

6. The system of claim 4, wherein the control module is configured to prompt the second user to determine if the second user would like to continue observing the media in response to the focus level of the second user indicating the second user is attentive or present.

7. The system of claim 6, wherein the control module is configured to:
receive feedback from the second user; and
continue playing or pause the media output by the media output module in response to the feedback from the second user.

8. The system of claim 1, wherein the control module is configured to determine whether the media is pauseable prior to detecting whether the user is observing the media.

9. The system of claim 1, wherein the one or more sensors include at least one of an occupancy sensor, a motion sensor, a camera, a radar sensor, a biometric sensor, and a microphone.

10. The system of claim 1, wherein:
the one or more detected characteristics include one or more of a location of the user, an eye focus of the user, a paired output device, and speaker tuning; and
the control module is configured to detect whether the user is observing media output by the media output module based on one or more of the location of the user, the eye focus of the user, the paired output device, and the speaker tuning.

11. The system of claim 1, wherein:
the one or more detected characteristics include one or more of an eye focus of the user, movement of the user, biometric data specific to the user, and environment data; and
the control module is configured to determine the focus level of the user based on one or more of the eye focus of the user, the movement of the user, the biometric data specific to the user, and the environment data.

12. The system of claim 1, wherein:
the focus level of the user is one of a plurality of focus states; and
the control module is configured to determine a focus state from the plurality of focus states for the user based on the at least one of the detected characteristics, and set the at least one bookmark moment in response to the determined focus state.

13. The system of claim 12, wherein the plurality of focus states include a focused and observable state, an inattentive and observable state, and a non-observable state.

14. The system of claim 1, wherein:
the set bookmark moment is one of a plurality of set bookmark moments for the media; and
the one or more resumption options include reverting to the set bookmark moment, implementing a contextual search of the plurality of set bookmark moments or the media, and displaying a menu of the plurality of bookmark moments.

15. The system of claim 1, wherein the one or more sensors and the control module are positioned in a vehicle.

16. A method for controlling media, the method comprising:
receiving, from at least one sensor, one or more characteristics;
detecting whether a user is observing media output by a media output module based on at least one of the characteristics;
in response to detecting the user is observing the media output by the media output module, determining a focus level of the user based on at least one of the characteristics;
setting at least one bookmark moment with respect to the media based on the focus level of the user; and
outputting one or more resumption options for the user with respect to the media, the one or more resumption options including reverting to the at least one bookmark moment.

17. The method of claim 16, wherein:
the method further includes pausing the media output by the media output module in response to the focus level of the user indicating the user is inattentive or absent; and
outputting the one or more resumption options for the user includes outputting the one or more resumption options in response to receiving a user request to resume the media.

18. The method of claim 17, wherein:
the user is a first user; and
the method further includes:
determining a focus level of a second user observing the media based on at least one of the characteristics; and
prompting the second user to determine if the second user would like to continue observing the media in response to the focus level of the second user indicating the second user is attentive or present.

19. A vehicle system for controlling media, the vehicle system comprising:
one or more sensors positioned in a vehicle, the one or more sensors configured to detect one or more characteristics; and
a control module in communication with the one or more sensors, the control module configured to:
detect whether a user is observing media output by a media output module based on at least one of the detected characteristics;
in response to detecting the user is observing the media output by the media output module, determine a focus state from a plurality of focus states for the user based on at least one of the detected characteristics;
set at least one bookmark moment with respect to the media based on the determined focus state for the user; and
output one or more resumption options for the user with respect to the media, the one or more resumption options including reverting to the at least one bookmark moment.

20. The vehicle system of claim 19, wherein the plurality of focus states include a focused and observable state, an inattentive and observable state, and a non-observable state.
